(19) United States
(12) Patent Application Publication
(10) Pub. No.: US 2022/0133547 A1
(43) Pub. Date: May 5, 2022

(54) Tough Aid Industrial Grade Medical Bandaged

(71) Applicant: Andrew P. Jackson, Wharton, TX (US)

(72) Inventor: Andrew P. Jackson, Wharton, TX (US)

(21) Appl. No.: 17/085,262

(22) Filed: Oct. 30, 2020

(51) Int. Cl. A61F 13/02

(52) U.S. Cl. CPC A61F 13/0246; A61F 13/0206

(57) ABSTRACT

The present invention discloses a tough medical bandage containing a medicament material capable of aiding in the healing of injuries in harsh/hazardous environments.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field of the Invention

The present invention relates in general to a bandage for covering a particular part of a. body such that a desired area can be allowed to heal even in heavy industrial environment.

### Description of the Related Art

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof to anything stated or implied therein or interred thereupon.

Overtime Band-Aid™ type bandages have had a dry or dry antiseptic coating infused into the gauze. The drawback is when a person with a wound places dry bandages on it, sweating between the gauze and the wound naturally occurs to a degree, depending on the severity of the said wound. Making it not as comfortable or as effective fighting infection.

Adhesive bandages, the term being used herein to designate not only the elongated strips, but also the round spots and the relatively square patches are formed of a backing sheet which may be cloth or plastic coated on one side with an adhesive and having a dressy dressing, in which case they are usually as large as the endwise adhesive areas.

Conventional bandages now available are, there may be mentioned surgical dressings, first-aid type bandages, such as Band-Aids, military medical bandages, operational bandages, such as swabs, and the like. In general, these prior art bandages comprise a layer of absorbent material, such as gauze, which is applied to the injured area of the skin by means of an adhesive material associated therewith which is applied to an adjacent uninjured area. In use, a medicant is usually initially applied to the injury to aid the healing process and the bandage or dressing is then applied thereover.

Many proposals have been made heretofore to provide bandages or dressings of the above-mentioned type wherein the absorbent portion to be applied to the injury is further provided with the medicinal material adhered directly thereto. The advantage of a dressing or bandage construction of this type, of course, resides in the elimination of the intermediate step of applying the medicant to the injury. A further advantage is realized by the elimination of the possibility that the medicinal, which is often liquid, will be lost by run-oh or leakage.

Certain disadvantages, however, have been found to be associated with dressings or bandages of the type provided with a medicant adhered thereto. The most prevalent of these disadvantages is the realization that medicants cannot be adhered for application in the liquid form by this method and, therefore, such bandages or dressings have been limited to uses involving materials which can be adhered as solids. A second disturbing factor regarding such medicaments resides in the ever present possibility of contamination of the medicinal materials with the obvious resulting consequences. It is apparent therefore that a distinct need is present in the art for the provision of a medicine-containing dressing or bandage which is not subject to the disadvantages of similar dressings or bandages now known to the prior art.

None of the prior art methods have been found to be completely suitable to meet these needs and are cumbersome. The present invention provides such a method and the overall combination of these features is nowhere disclosed in the prior art cited above which appears to be representative of the general art in this area although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, and abstract as a whole.

An important object of the invention is to provide an adhesive bandage with facing strips which may be removed from the bandage with minimum danger of contaminating the dressing.

Another objective of the present invention resides in the provision of medical bandages or dressings wherein the areas thereof to be applied to the injury are provided with the appropriate mendicant contained within a bandage which is wider and would be completely warped around. the area, completely sealing off the wound.

In an alternative embodiment, of the present invention to overcome the above-mentioned shortcomings of known bandages for surgical dressings and the like and to provide a bandage which will not slip out of place when applied.

It is also an object of the invention to provide a bandage which wear for a longer time and which can be washed a number of times without losing its shape or elasticity.

Another significant embodiment of the present invention is a bandage for the use in heavy industrial environment where the user would not to have to worry about further damage to the injury and the bandage will would hold up much better than anything on the market in harsh/hazardous environments

It is an object of the invention to provide a bandage that can be enlarged in scale to create very effective pressure bandages for larger wounds

One object of the invention is to provide an improved bandage construction.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a full view of the invention.

FIG. 2 is a side view of the invention.

FIG. 3 is a top view of the invention.

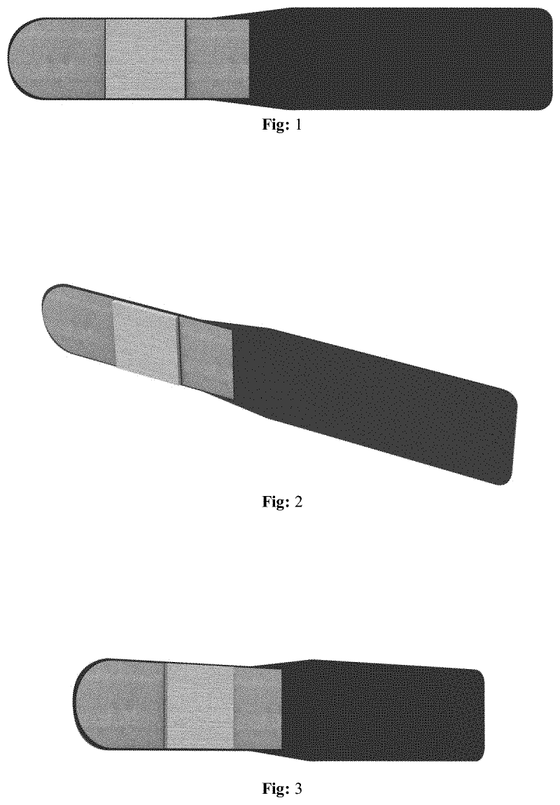

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

Unless otherwise defined, all terms (including technical and scientific terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques are disclosed. Each of these has individual benefit and each can also be used in conjunction with one or more, or in some cases all, of the other disclosed techniques. Accordingly, for the sake of clarity, this description will refrain from repeating possible combination in an unnecessary fashion. Nevertheless, the specifications and claim/s should be read with the understanding that such combinations are entirely within the scope of the invention and the claim/s.

The method of the present invention is that it is an improved medical dressing or bandage for use in treating injuries may be provided by the adherence to absorbent dressings of the medical bandage or dressing type containing chemical agents which, when the capsules are ruptured, are released to effectively aid in the healing of injuries and burns, relieve pain, coagulate blood, etc., in association with the absorbing qualities of the bandage or dressing substrate and can be used in tougher industrial environments so it doesn't tear off easily.

As mentioned, the bandage or dressing may basically comprise any of the various types of such materials now on the market including surgical dressings, adhesive bandages, operational dressings (i.e., swabs), military bandages, etc., as the invention is considered applicable to all types of such materials.

According to the concepts embodied in the present invention, the core materials which are encapsulated for use on the medical dressings or bandages may be generally described as medicinal formulations for the treatment of heat, e.g. burns, etc., pain relievers, medicinals employed in treating injuries, e.g. antiseptics, antibiotics, blood coagulants, disinfectants, local anesthetics and the like, or any material considered to be of value in administering medicinals to injuries.

It is now seen that there is herein provided an improved transparent bandage which accomplishes all of the objects and advantages of the instant invention and others, including many advantages of great practical utility and commercial importance.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1) Medical dressing comprising a dressing substrate that would bond to the skin like a normal bandage, the medical gauze pad attached to the bottom of the bandage would then be placed onto of the wound thus completely sealing off the wound.

2) The invention would hold up much better than anything in harsh/hazardous environments.

3) A medical dressing construction according to claim 1 wherein the dressing is of the adhesive bandage type that would wrap tightly in harsh/hazardous environments.
